SEQUENCE LISTING (4 sequences)

SEQ ID NO: 1 - DNA, 781 bp, Artificial Sequence
  Synthetic primer
atggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccatggagcacctggagcttatcccggagcacctgcacctggagtctacccagggccacccagcggccctggggcctacccatcttctggacagccaagtgcccccggagcctaccctgccactggcccctatggcgcccctgctgggccactgattgtgccttataacctgcctttgcctgggggagtggtgcctcgcatgctcataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataatctgaaaggggcagattaaaaaaaaaaa

SEQ ID NO: 2 - protein, 240 aa, Artificial Sequence
  Synthetic peptide
MADNFSLHDALSGSGNPNPQGWPGAWGNQPAGAGGYPGASYPGAYPGQAPPGAYPGQAPPGAYPGAPGAYPGAPAPGVYPGPPSGPGAYPSSGQPSATGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDI

SEQ ID NO: 3 - DNA, 460 bp, Artificial Sequence
  Synthetic primer
ggcgcccctgctgggccactgattgtgccttataacctgcctttgcctgggggagtggtgcctcgcatgctcataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataatctgaaaggggcagattaaaaaaaaaaa

SEQ ID NO: 4 - protein, 143 aa, Artificial Sequence
  Synthetic peptide
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

(19) United States
(12) Patent Application Publication
(10) Pub. No.: US 2015/0157691 A1
(43) Pub. Date: Jun. 11, 2015

(54) Galectin-3 to Treat Ovarian Cancer

(71) Applicant: Texas Tech University System, Lubbock, TX (US)

(72) Inventors: Maurizio Chiriva-Internati, Lubbock, TX (US); Jose A. Figueroa, Lubbock, TX (US); Everardo Cobos, Lubbock, TX (US)

(21) Appl. No.: 14/561,981

(22) Filed: Dec. 5, 2014

Related U.S. Application Data
(60) Provisional application No. 61/912,241, filed on Dec. 5, 2013.

(51) Int. Cl. A61K 38/17; G01N 33/561; A61K 31/337

(52) U.S. Cl. CPC A61K 38/1732; A61K 31/337; G01N 33/561; A61K 48/00

(57) ABSTRACT

The present invention includes a method for the treatment of an advanced ovarian cancer, comprising: identifying a patient with advanced ovarian cancer; and administering to the patient an effective amount of truncated, dominant negative form of Galectin-3 sufficient to reduce the advanced ovarian cancer. In certain aspects, the truncated, dominant negative form of Galectin-3 is provided in an amount sufficient to reduce at least one of growth, motility, invasion, angiogenesis, or prevents Akt/NF-κB activation of the ovarian cancer.

### CROSS-REFERENCE TO RELATED APPLICATIONS

This application claims priority based on U.S. Provisional Application No. 61/912,241, filed Dec. 5, 2013. The contents of which is incorporated by reference in its entirety.

### TECHNICAL FIELD OF THE INVENTION

The present invention relates in general to the field of treatments for ovarian cancer, and more particularly, to the use of Galectin-3 to suppress drug resistance, motility, invasion, and/or the angiogenic potential of ovarian cancer.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Dec. 5, 2014, is named TECH1098_SeqList.txt and is 6 kilobytes in size.

### BACKGROUND OF THE INVENTION

Without limiting the scope of the invention, its background is described in connection with ovarian cancer.

Ovarian cancer (OC) ranks first for mortality rates among gynecologic malignancies (1). Despite largely investigated, OC origin and pathogenesis are still poorly understood, significantly limiting the development of new, OC-tailored drugs (2). Currently, first-line treatment is based on a combination of surgery and chemotherapy. Response rates and complete response rates to carboplatin and paclitaxel are initially seen in more than 80% and 60% of patients, respectively (3). Unfortunately, after a first complete remission the majority of patients recur (75%). The progressive development of drug resistance and the accumulation of toxicities dramatically limit further treatment options. Although promising improvements in overall survival rates have been described through the use of paclitaxel and intraperitoneal chemotherapy (4), relapsed and advanced OC is still incurable (5). The development of drug resistance is tightly associated to the acquirement of an invasive and motile phenotype during tumor progression (6, 7), which results in tumor spread, induction of angiogenesis (8, 9), and is correlated to adverse prognosis (10). Tumor-induced neo-angiogenesis is also one of the main causes of increased tumor burden (11), and in vivo studies have clearly proven that tumor-induced endothelial cell recruitment and differentiation are critical in OC progression (12, 13). Therefore, there is an evident need of novel OC-tailored drugs, allowing to improve the outcome of available treatments without exacerbating related toxicities, and to reduce tumor spread and associated angiogenesis.

Galectins are S-type lectins that bind β-galactose-containing glycoconjugates (14). Since the discovery of the first galectin in animal cells in 1975 (15), fifteen mammalian galectins have been isolated. They regulate different biological processes such as cell adhesion, regulation of growth, apoptosis, tumor development and progression (16). Accumulating evidences report multiple roles for Galectins in OC (17). Among them, Galectin-3 is an attractive target, since it is involved in many features of tumor progression such as adhesion, proliferation, and metastasis (18, 19). Indeed, Galectin-3 was shown to promote OC drug resistance (20). Additionally, it was reported that OC metastasis (21) and resistance to paclitaxel (22) are promoted by the Akt/NF-κB axis, which is known to depend on Galectin-3 in multiple myeloma (23).

U.S. Pat. No. 6,770,622, issued to Jarvis, et al., is directed to an N-terminally truncated Galectin-3 for use in treating cancer. Briefly, this patent teaches a composition having an effective amount of N-terminally truncated Galectin-3 in a pharmaceutically acceptable carrier. Also provided by the present invention is a method of treating cancer by administering to a patient in need of such treatment an effective amount of N-terminally truncated Galectin-3 in a pharmaceutically acceptable carrier. Data is provided that shows the treatment of breast cancer cells in a mouse model system.

International Patent Publication WO2012135528 A2, by the present inventors, is directed to the use of Galectin-3c in combination therapy for human cancer, in which Galectin-3C was used in combination with a proteosome inhibitor, the combination having a pharmacologic activity greater than the expected additive effect of its individual components. Other embodiments of the invention provide compositions of Galectin-3C with a proteasome inhibitor capable of reducing or overcoming resistance that develops to the proteasome inhibitor or reducing the adverse side effects from the proteasome inhibitor through increasing the therapeutic efficacy of lower doses.

Others have published results that show that Galectin-3C was used to treat multiple myeloma, including the use of Galectin-3C in conjunction with the chemotherapeutic Bortezomib, Mirandola, L., et al. “Galectin-3C Inhibits Tumor Growth and Increases the Anticancer Activity of Bortezomib in a Murine Model of Human Multiple Myeloma” Plos One, July 2011|Volume 6|Issue 7|e21811.

### SUMMARY OF THE INVENTION

In one embodiment, the present invention includes a method for the treatment of an advanced ovarian cancer, comprising: identifying a patient with advanced ovarian cancer; and administering to the patient an effective amount of truncated, dominant negative form of Galectin-3 sufficient to reduce the advanced ovarian cancer. In one aspect, the truncated, dominant negative form of Galectin-3 is administered by intravenous or intraperitoneal route. In another aspect, the ovarian cancer is drug resistant. In another aspect, the ovarian cancer is multiple-drug resistant. In another aspect, the ovarian cancer is an advanced, refractory ovarian cancer. In another aspect, the amount of the truncated, dominant negative form of Galectin-3 is sufficient to reduce at least one of growth, motility, invasion, angiogenesis, or prevents Akt/NF-κB activation in ovarian cancer cells. In another aspect, the method further comprises the addition of paclitaxel to the treatment. In another aspect, the truncated, dominant negative form of Galectin-3 is provided as a nucleic acid vector having SEQ ID NO.:3, and expressed as SEQ ID NO.: 4. In another aspect, the truncated, dominant negative form of Galectin-3 is provided as a polypeptide having SEQ ID NO.:4. In another aspect, the truncated, dominant negative form of Galectin-3 is provided as a nucleic acid in an expression vector that expresses the truncated, dominant negative form of Galectin-3 upon entry into a cell.

The present invention also includes a method for the treatment of an advanced, refractory ovarian cancer, comprising: identifying a patient with advanced, refractory ovarian cancer; and administering to the patient an effective amount of a truncated, dominant negative form of Galectin-3, in combination with paclitaxel, in free or pharmaceutically acceptable salt form to reduce or eliminate the ovarian cancer. In another aspect, the truncated, dominant negative form of Galectin-3 is administered by intravenous or intraperitoneal route. In another aspect, the ovarian cancer is drug resistant. In another aspect, the ovarian cancer is multiple-drug resistant. In another aspect, the amount of the truncated, dominant negative form of Galectin-3 is sufficient to reduce at least one of growth, motility, invasion, angiogenesis, or prevents Akt/NF-κB activation in ovarian cancer cells. In another aspect, the truncated, dominant negative form of Galectin-3 is provided as a nucleic acid vector having SEQ ID NO.:3, and expressed as SEQ ID NO.: 4. In another aspect, the truncated, dominant negative form of Galectin-3 is provided as a polypeptide having SEQ ID NO.:4. In another aspect, the truncated, dominant negative form of Galectin-3 is provided as a nucleic acid in an expression vector that expresses the truncated, dominant negative form of Galectin-3 upon entry into a cell.

Yet another embodiment of the present invention includes a method of determining the effectiveness of a candidate drug believed to be useful in treating ovarian cancer, the method comprising: (a) measuring from tissue suspected of having ovarian cancer from a set of patients; (b) administering a candidate drug to a first subset of the patients, and a placebo to a second subset of the patients, wherein the candidate substance is at least one of a truncated or a dominant negative form of Galectin-3; (c) repeating step (a) after the administration of the candidate drug or the placebo; and (d) determining if the candidate drug reduces at least one of the number or proliferation of ovarian cancer cells, reduces at least one of growth, motility, invasion, or angiogenesis caused by ovarian cancer cells, or prevents Akt/NF-κB activation of the ovarian cancer that is statistically significant as compared to any reduction occurring in the second subset of patients, wherein a statistically significant reduction indicates that the candidate drug is useful in treating the ovarian cancer.

### BRIEF DESCRIPTION OF THE DRAWINGS

For a more complete understanding of the features and advantages of the present invention, reference is now made to the detailed description of the invention along with the accompanying figures and in which:

FIG. 1 shows four graphs showing a flow-cytometry analysis of Galectin-3 expression by OC cells. Exponentially growing human (SKOV-3, Pt1, Pt2) or murine (ID8) OC cells were harvested and stained for Galectin-3, as described in Methods. The panel shows overlay histograms of anti-Galectin-3 antibody (bold gray lines), and isotype-control (thin black lines). Mean fluorescence intensity (MFI) representative of three independent analysis with similar results is indicated.

FIGS. 2A and 2B show Gal-3C effects on OC cell viability. FIG. 2A shows a dose-response curve using increasing Gal-3C concentrations. Error bars represent the 95% confidence interval. FIG. 2B shows OC cell viability was measured after 48-h treatment with 10 μg/mL Gal-3C, 8 nM paclitaxel, or combined treatments (G+P). Error bars represent the 95% confidence interval. *=one-way ANOVA and Tukey's post-test p<0.05; **=p<0.01; ***=p<0.001.

FIGS. 3A and 3B show Western blots for the evaluation of the Akt/NF-kB pathway activation. Total protein lysates from differently treated OC cells (columns) were analyzed by Western blotting after 48-h treatments using the antibodies raised against the indicated antigens (rows). A representative result was selected from three independent experiments with similar outcomes.

FIGS. 4A and 4B show invasion and migration assays. OC cells were tested in Matrigel™ invasion assay (FIG. 4A) or Transwell™ migration assay (FIG. 4B) in the presence of 10 μg/mL Gal-3C, 8 nM paclitaxel, or combined treatments (G+P). Bars represent the mean of invading/migrating cells out of three independent experiments. Error bars indicate 95% confidence interval. *=one-way ANOVA and Tukey's post-test p<0.05; **=p<0.01; ***=p<0.001, for drug-treated versus control cells. No statistically significant differences were detected between treatments.

FIG. 5 shows a graph of a vascular endothelial cell migration assay. The ability of the 48-h OC conditioned medium to recruit HUVEC cells was tested in a Transwell™ migration assay. Bars represent the mean of three independent experiments (error bars, 95% confidence interval). *=one-way ANOVA and Tukey's post-test (versus control c.m.) p<0.05; ***=p<0.001.

FIGS. 6A and 6B show the results of an in vitro angiogenesis assay. The conditioned media of 48-h treated OC cells was tested for its ability to induce the formation of tubules by HUVEC cells. Representative pictures of three independent experiments are shown in FIG. 6A. The degree of tubule formation was assayed as indicated in Methods and presented in FIG. 6B as the mean number of branching points (bars)±95% confidence interval. *=one-way ANOVA and Tukey's post-test (versus control c.m.) p<0.05; **=p<0.01; ***=p<0.001.

FIG. 7 is a graph that shows an analysis of αvβ3 integrin clusters in vascular endothelial cells. Following 48-h treatments of OC with 10 μg/mL Gal-3C, 8 nM paclitaxel, or combined treatments (G+P), the conditioned medium (20% V/V) was added to HUVEC cells, and the clusters of αvβ3 integrins were displayed using a Leica TCS-SL Confocal Spectral Microscope System. Representative pictures of three independent experiments with similar results are shown. White triangles indicate clusters.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The terms “administration of” or “administering a” compound should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” means the amount of the subject compound that will elicit the biological or medical response of a tissue, system, animal or human that is being sought by the researcher, veterinarian, medical doctor or other clinician. As used herein, the term “treatment” refers to the treatment of the mentioned conditions, particularly in a patient who demonstrates symptoms of the disease or disorder.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “resistant” or “refractory” when referring to a cancer means that the cancer cells are no longer susceptible to a particular chemotherapy or other treatment, e.g., radiation, and thus the cancers do not respond to previous anticancer therapy or treatment. The present invention, when used to target ovarian cancer, was demonstrated to successfully treat a resistant or refractory cancer such that the resistant or refractory symptoms or conditions are prevented, minimized or attenuated during and/or after anticancer treatment, when compared to that observed in the absence of the treatment described herein. The minimized, attenuated or prevented refractory conditions can be confirmed by clinical markers contemplated by the artisan in the field. In one non-limiting example, successful treatment of refractory or resistant cancer shall be deemed to occur when at least 10, 20, 30, 40, 50, 60, 70, 80, 90, 95, 96, 97, 98, 99 or 100% inhibition of cancer cell proliferation, decrease in tumor growth, and/or preventing recurrence is obtained when compared to that observed in the absence of the treatment of the present invention.

The term “truncated, dominant negative form of Galectin-3” refers to the nucleotides essentially as set forth (SEQ ID NO. 3) or the amino acid sequence essentially as set forth (SEQ ID NO 4).

The terms “a sequence essentially as set forth in SEQ ID NO. (#)”, “a sequence similar to”, “nucleotide sequence” and similar terms, with respect to nucleotides, refers to sequences that substantially correspond to any portion of the sequence identified herein as SEQ ID NO.: 1. These terms refer to synthetic as well as naturally-derived molecules and includes sequences that possess biologically, immunologically, experimentally, or otherwise functionally equivalent activity, for instance with respect to hybridization by nucleic acid segments, or the ability to encode all or portions of a truncated, dominant negative form of Galectin-3. Naturally, these terms are meant to include information in such a sequence as specified by its linear order.

The term “gene” is used to refer to a functional protein, polypeptide or peptide-encoding unit. As will be understood by those in the art, this functional term includes genomic sequences, cDNA sequences, or fragments or combinations thereof, as well as gene products, including those that may have been altered by the hand of man. As claimed herein the recombinant portions of the truncated, dominant negative form of Galectin-3 refer to cDNA sequences. Purified genes, nucleic acids, protein and the like are used to refer to these entities when identified and separated from at least one contaminating nucleic acid or protein with which it is ordinarily associated.

As used herein, the term “vector” is used in reference to nucleic acid molecules that transfer DNA segment(s) from one cell to another. The vector may be further defined as one designed to propagate the truncated, dominant negative form of Galectin-3 sequences, or as an expression vector that includes a promoter operatively linked to the truncated, dominant negative form of Galectin-3 sequence, or one designed to cause such a promoter to be introduced. The vector may exist in a state independent of the host cell chromosome, or may be integrated into the host cell chromosome

The term “host cell” refers to cells that have been engineered to contain nucleic acid segment encoding a truncated, dominant negative form of Galectin-3, or altered segments, whether archeal, prokaryotic, or eukaryotic. Thus, engineered, or recombinant cells, are distinguishable from naturally occurring cells that do not contain recombinantly introduced genes through the hand of man.

The term “altered”, or “alterations” or “modified” with reference to nucleic acid or polypeptide sequences is meant to include changes such as insertions, deletions, substitutions, fusions with related or unrelated sequences, such as might occur by the hand of man, or those that may occur naturally such as polymorphisms, alleles and other structural types. Alterations encompass genomic DNA and RNA sequences that may differ with respect to their hybridization properties using a given hybridization probe. Alterations of polynucleotide sequences for the truncated, dominant negative form of Galectin-3, or fragments thereof, include those that increase, decrease, or have no effect on functionality. Alterations of polypeptides refer to those that have been changed by recombinant DNA engineering, chemical, or biochemical modifications, such as amino acid derivatives or conjugates, or post-translational modifications.

Ovarian cancer is the most deadly gynecologic malignancy worldwide. Because the pathogenesis of ovarian cancer is still incompletely understood, and there are no available screening techniques for early detection, patients are mostly diagnosed with advanced disease, which results ultimately fatal. In the effort to develop innovative effective chemotherapies, the present inventors demonstrate herein that the truncated, dominant negative form of Galectin-3, is effective in significantly reducing the growth, motility, invasion, and angiogenetic potential of cultured OC cell lines and primary cells established from OC patients. Overall, these findings clearly show that Galectin-3C is a new compound for effective adjuvant therapies in advanced and refractory ovarian cancer. Galectin-3 is encoded by a single gene, LGALS3, located on chromosome 14, locus q21-q22, having cDNA sequence:

(SEQ ID NO. 1)
ATGGCAGACAATTTTTCGCTCCATGATGCGTTATCTGGGTCTGGAAACC
CAAACCCTCAAGGATGGCCTGGCGCATGGGGGAACCAGCCTGCTGGGGC
AGGGGGCTACCCAGGGGCTTCCTATCCTGGGGCCTACCCCGGGCAGGCA
CCCCCAGGGGCTTATCCTGGACAGGCACCTCCAGGCGCCTACCATGGAG
CACCTGGAGCTTATCCCGGAGCACCTGCACCTGGAGTCTACCCAGGGCC
ACCCAGCGGCCCTGGGGCCTACCCATCTTCTGGACAGCCAAGTGCCCCC
GGAGCCTACCCTGCCACTGGCCCCTATGGCGCCCCTGCTGGGCCACTGA
TTGTGCCTTATAACCTGCCTTTGCCTGGGGGAGTGGTGCCTCGCATGCT
CATAACAATTCTGGGCACGGTGAAGCCCAATGCAAACAGAATTGCTTTA
GATTTCCAAAGAGGGAATGATGTTGCCTTCCACTTTAACCCACGCTTCA
ATGAGAACAACAGGAGAGTCATTGTTTGCAATACAAAGCTGGATAATAA
CTGGGGAAGGGAAGAAAGACAGTCGGTTTTCCCATTTGAAAGTGGGAAA
CCATTCAAAATACAAGTACTGGTTGAACCTGACCACTTCAAGGTTGCAG
TGAATGATGCTCACTTGTTGCAGTACAATCATCGGGTTAAAAAACTCAA
TGAAATCAGCAAACTGGGAATTTCTGGTGACATAGACCTCACCAGTGCT
TCATATACCATGATATAATCTGAAAGGGGCAGATTAAAAAAAAAAA

Galectin-3 has an amino acid sequence of:

(SEQ ID NO. 2)
MADNFSLHDA LSGSGNPNPQ GWPGAWGNQP AGAGGYPGAS YPGAYPGQAP PGAYPGQAPP
GAYPGAPGAY PGAPAPGVYP GPPSGPGAYP SSGQPSATGA YPATGPYGAP AGPLIVPYNL
PLPGGVVPRM LITILGTVKP NANRIALDFQ RGNDVAFHFN PRFNENNRRV IVCNTKLDNN
WGREERQSVF PFESGKPFKI QVLVEPDHFK VAVNDAHLLQ YNHRVKKLNE ISKLGISGDI
DLTSASYTMI.

Galectin-3C has cDNA sequence

(SEQ ID NO.: 3)
GGCGCCCCTGCTGGGCCACTGATTGTGCCTTATAACCTGCCTTTGCCTG
GGGGAGTGGTGCCTCGCATGCTCATAACAATTCTGGGCACGGTGAAGCC
CAATGCAAACAGAATTGCTTTAGATTTCCAAAGAGGGAATGATGTTGCC
TTCCACTTTAACCCACGCTTCAATGAGAACAACAGGAGAGTCATTGTTT
GCAATACAAAGCTGGATAATAACTGGGGAAGGGAAGAAAGACAGTCGGT
TTTCCCATTTGAAAGTGGGAAACCATTCAAAATACAAGTACTGGTTGAA
CCTGACCACTTCAAGGTTGCAGTGAATGATGCTCACTTGTTGCAGTACA
ATCATCGGGTTAAAAAACTCAATGAAATCAGCAAACTGGGAATTTCTGG
TGACATAGACCTCACCAGTGCTTCATATACCATGATATAATCTGAAAGG
GGCAGATTAAAAAAAAAAA

Galectin-3C has amino acid sequence

(SEQ ID NO. 4)
GAPAGPLIVPYNLPLPGGVVPRMLITILGTVKPNANRIALDFQRGNDVA
FHFNPRFNENNRRVIVCNTKLDNNWGREERQSVFPFESGKPFKIQVLVE
PDHFKVAVNDAHLLQYNHRVKKLNEISKLGISGDIDLTSASYTMI

The present inventors have previously shown that Galectin-3 blockade hampered tumor growth and spread in breast cancer animal models (24), and sensitized multiple myeloma to bortezomib, reducing malignant cell ability to induce angiogenesis (25). Regardless, the present inventors tested the outcome of Galectin-3 inhibition in ovarian cancer (OC) cell lines and primary cells derived from OC patients. Galectin-3 is unique in the galectin family, since it displays a carboxyl-terminal carbohydrate recognition domain (CRD, which binds to β-galactosides), and an amino-terminal domain that is critical for Galectin-3 multivalent behavior (25). Alone, the CRD is incapable of the cooperative binding that characterizes the intact lectin, since the N-terminal domain enables the CRD to cross-link proteins containing β-galactosides, modulating cell adhesion and signaling. Thus, the inventors used a truncated, dominant-negative form of Galectin-3, termed Galectin-3C (Gal-3C), to block Galectin-3 in OC cells. The truncated Galectin-3 used in this study includes the last 143 carboxy-terminal amino acid residues of human Galectin-3. In certain embodiments, the truncated Galectin-3 used in this study consists essentially of the last 143 carboxy-terminal amino acid residues of human Galectin-3, and in one embodiment consists of the last 143 carboxy-terminal amino acid residues of human Galectin-3. Since it lacks the N-terminal domain, the carbohydrate binding abilities are preserved, but not the cooperative binding properties. Therefore, Gal-3C could act as a dominant negative inhibitor of Galectin-3 (24, 25).

It is shown herein that Gal-3C, alone or in combination with paclitaxel, reduces OC cell growth, invasion, and migration in vitro, prevents Akt/NF-κB activation, and hampers tumor cell angiogenic potential. The inventors further provide evidence that Galectin-3 is a target for effective adjuvant therapies in advanced OC.

Reagents and Drugs. Paclitaxel was purchased from Ben Venue Labs (Bedford, Ohio, USA). Gal-3C was prepared as previously described (25). Antiphospho-IKKα/β (Ser176/180), anti-IKKα/β, anti-phospho-IKBα (Ser32), anti-IKBα, anti-phospho-NF-κB p65 (Ser536), anti-NF-κB, anti-phospho-Akt (Ser473) and anti-Akt antibodies were from Cell Signaling Technology (Danvers, Mass. USA).

Cells. The OC cell lines used in this study were SKOV-3 and ID8 (American Type Culture Collection, Manassas, Va., USA), while primary epithelial OC cells from two patients were obtained with approval from the Texas Tech University HSC IRB (L-Micro Study, IRB NUMBER: L04-095), and the patients' written informed consent. All OC cells were cultured in RPMI-1640 medium supplemented with 10% fetal bovine serum (FBS, Thermo Scientific, Rockford, Ill., USA) in 5% CO₂ atmosphere at 37° C. Human Umbilical Vein Endothelial Cells (HUVEC, American Type Culture Collection) were maintained in EGM-2 medium supplemented with endothelial cells growth factors (Lonza, Houston, Tex., USA) and were used within 10 passages.

Flow cytometric analysis. The expression of Galectin-3 was analyzed by flow cytometry. Briefly, OC single-cell suspensions were distributed into 12×75 mm flow cytometry tubes (1×10₅ cells/tube). Cells were incubated with 1 μg/mL mouse monoclonal anti-human/mouse Galectin-3 IgG1 (LifeSpan Biosciences, Inc., Seattle, Wash., USA, clone B2C10) in 20 μL PBS (pH=7.4) for 1 hour on ice. 1 μg/mL mouse IgG1 was used as isotype matched control (Santa Cruz Biotechnology, Inc., Santa Cruz, Calif., USA). Then, cells were washed three times with ice-cold PBS (0.2 mL), and incubated with 0.2 μg/mL FITC-conjugated rat anti-mouse Ig (BD Biosciences, San Jose, Calif., USA) in 20 μL PBS for 1 hour on ice in the dark. Cells were analyzed with a FACScan flow-cytometer (BD Biosciences) after washing two times with 0.3 mL ice-cold PBS.

Cell viability. Cell proliferation was assessed with a ViaLight Plus Cell Proliferation and Cytotoxicity BioAssay Kit (Lonza, Walkersville, Md., USA) according to the directions of the manufacturer. In brief, OC cells were seeded in 100 μL RPMI-1640 with 10% heat-inactivated FBS in 96-well plates (8×10³/well). Each drugs/drug combination was tested in triplicate. Luminescence was measured with a 1-s integrated setting in a Berthold luminometer.

Migration assays. 4×10⁵ cells were plated in 100 μL serum-free culture medium (supplemented with drugs or vehicle) in the top chambers of 24-well Transwell™ polycarbonate inserts (Corning Costar, NY, USA) with 8-μm pores. Complete culture medium (600 μL) was added to the bottom chamber. After incubation for 4 hours, cells on lower side of the filter were fixed, stained and counted as described (25).

For HUVEC cells, the assay was run using 5×10⁴ cells/well, and the lower chamber was filled with 600 μL EGM-2 medium supplemented with 20% V/V conditioned medium from OC cells (obtained after treating of OC cells with 10 μg/mL Gal-3C, 8 nM paclitaxel, or combined drugs). The experiments were run in triplicate and the results are expressed as the mean number of migrated cells in the presence of different stimuli.

Invasion assay. OC invasion potential was measured as described (25), using Transwell™ polycarbonate inserts (5-μm pore size).

Tubule formation assay. A capillary tubule formation assay was performed as previously described (25). Briefly, growth factor-reduced Matrigel™ (50 μL/well; Becton Dickinson) was added with 30 ng/mL recombinant basic fibroblastic growth factor (bFGF; R&D Systems) and incubated for 1 hour at 37° C. 2-h serum-starved HUVEC cells (5,000), were resuspended in 200 μL serum-free EGM-2 medium (Lonza) supplemented with 20% V/V conditioned medium from differently treated OC cells (obtained after 48-h treatment of OC cells with 10 μg/mL Gal-3C, 8 nM paclitaxel, the combination of both, or 0.6% V/V PBS as drug vehicle). HUVEC were then seeded onto the Matrigel™ and incubated at 37° C. and 5% CO₂ for 16 hours to allow tubule formation. The assays were run in triplicate, and microphotographs were taken using 40× and 20× objectives with an inverted X71 microscope (Olympus). Pictures were also used to count the number of branching points originated by HUVEC cells, as a measure of the degree of angiogenesis, by the Wimasis WimTube software (Wimasis GmbH, Munich, Germany). Statistically significant differences in the number of branching points were analyzed by the Kruskal-Wallis test followed by a Dunns post-test, to compare all the treatment groups with each other.

αvβ3 integrin clustering assay. HUVEC cells were cultured in 8-chamber slides coated with 10 μg/mL fibronectin (10⁴ cells/chamber). Cells were incubated with conditioned medium of SKOV-3 cells (obtained as described for the tubule formation assay) for 20 minutes to allow integrin clustering, then fixed with 4% WN buffered (pH=7.5) paraformaldehyde in PBS (10 minutes at 37° C.). Then, cells were incubated with 10 μg/mL anti-integrin αvβ3 antibody (R&D Systems, 1:200 dilution in PBS) for 1 hour at RT, followed by two washing steps in PBS (5 minutes each) prior to the addition of FITC-anti-mouse IgG (1:1000 dilution in PBS) for 1 hour at RT. Microphotographs of randomly selected fields were acquired at 60× magnification by confocal microscopy (Leica TCS-SL Confocal Spectral Microscope System, Leica Microsystems, Buffalo Grove, Ill., USA).

Western blotting. Cells were harvested and washed twice with PBS, then lysed (25), and resolved on 12% Bis-Tris (Bis(2-hydroxyethyl)-amino-tris(hydroxymethyl)-methane) poly-acrylamide gel (Invitrogen) and then electrotransferred onto 0.2 μm nitrocellulose membrane. After rinsing in PBS-Tween, the blot was incubated in protein-free blocking buffer (Pierce/Thermo) at 4° C. overnight, then incubated with the indicated primary antibodies (Cell Signaling Technology, Inc., Danvers, Mass. USA) diluted in blocking buffer for 2 hours at RT. After washing 5 times with PBS-Tween, the blot was incubated with matched horseradish peroxidase-coupled secondary antibodies, and developed with the ECL Western blotting Detection System from Perkin-Elmer Life Sciences (Boston, Mass., USA).

Statistical analyses. All of the data are expressed as mean values, and errors are expressed as the 95% confidence intervals. Results were analyzed using GraphPad Prism version 4.00 for Windows (GraphPad Software, San Diego, Calif., USA).

OC cell lines and cells from primary OC tumors expressed Galectin-3 at the cell surface. OC cell lines, SKOV-3 and ID8, and the OC cells from two patients (Pt1 and Pt2), were stained and analyzed by flow-cytometry to measure the expression of Galectin-3.

FIG. 1 shows the results obtained on unpermeabilized cells: all of the analyzed cells expressed Galectin-3 at the cell surface.

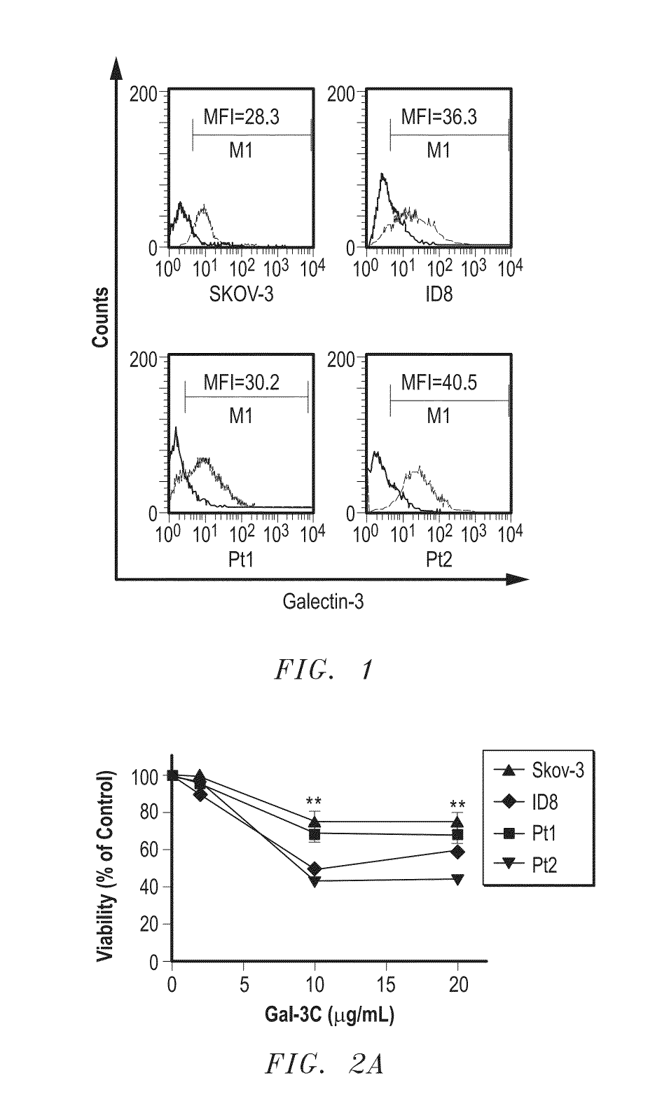

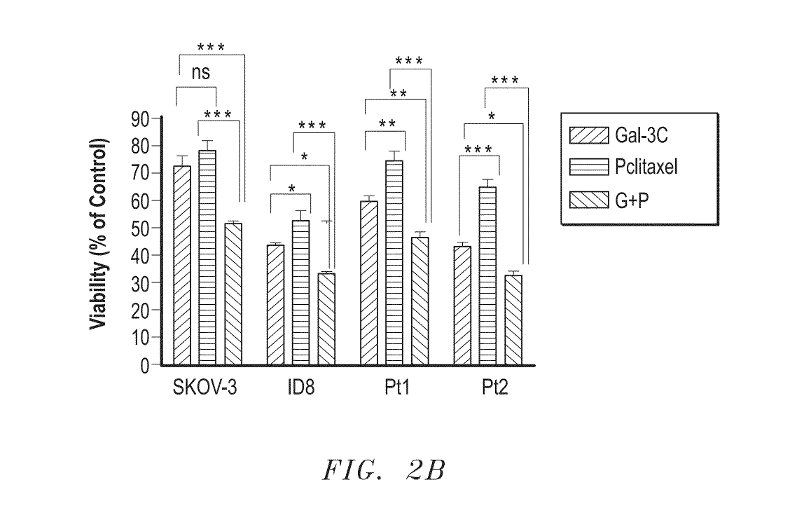

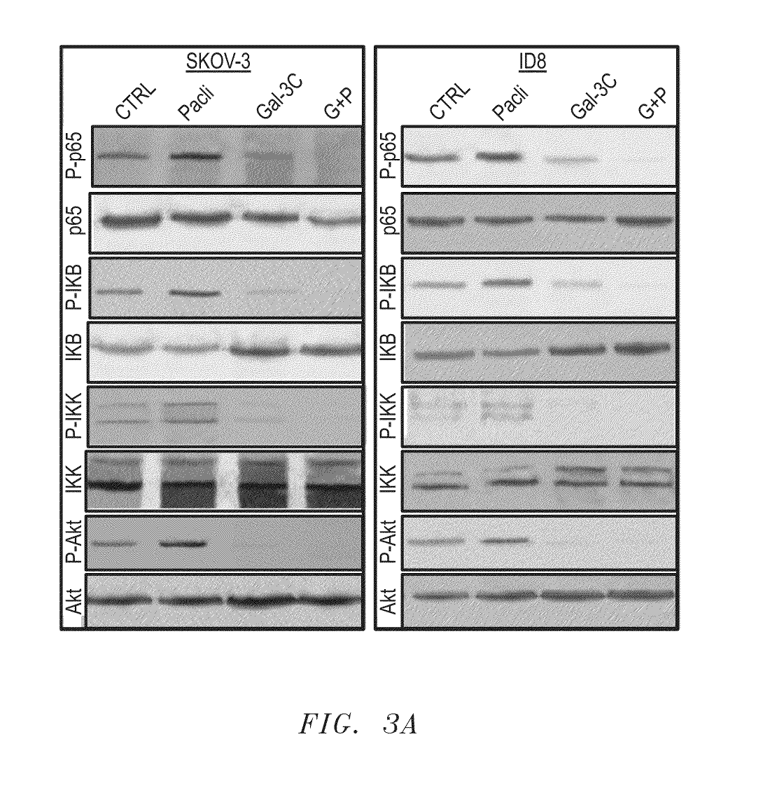

Administration of Gal-3C reduced OC cell viability when used as a single agent and improved the effects of paclitaxel. OC cells were treated with escalating concentrations of Gal-3C (0, 2, 10, and 20 μg/mL) for 48 hours, then viability was measure as described in methods, and expressed as percentage of control cells (treated with equal amounts of drug vehicle). FIG. 2A shows that Gal-3C significantly reduced cell viability starting at 10 μg/mL. On average, viability was reduced by 25% in SKOV-3, 50% in ID8, 29% in Pt1 and 57% in Pt2 cells. Then, in a separate set of experiments, OC cells were treated for 48 hours with 10 μg/mL Gal-3C, 8 nM paclitaxel, or combined 10 μg/mL Gal-3C+8 nM paclitaxel. Results (FIG. 2B) show that Gal-3C was more effective in reducing cell viability compared with paclitaxel in all cell lines except SKOV-3 (one-way ANOVA and Tukey's post-test p<0.05 for ID8, <0.01 for Pt1, 0.001 for Pt2). The combined treatment was more effective than single drugs in all of the tested cells (p<0.01), showing reduction of viability by 41% in SKOV-3, 67% in ID8, 53% in Pt1 and 68% in Pt2 cells (FIG. 2B).

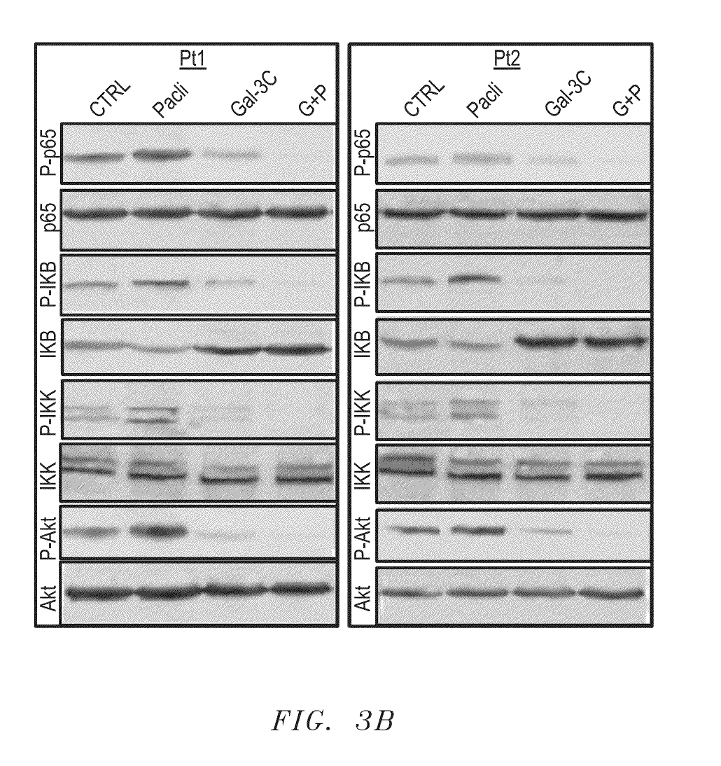

Gal-3C reversed the effect of paclitaxel on NF-κB activation by reducing the phosphorylation of Akt. OC cells treated for 48 hours with 10 μg/mL Gal-3C, 8 nM paclitaxel, or combined treatments as described above, then protein extracts were analyzed by Western blotting. FIGS. 3A and 3B show that paclitaxel increased the levels of phosphorylated, active NF-κB (P-p65), while contemporary administration of Gal-3C blocked such activation. Increased or decreased levels of p65 phosphorylation (P-p65) were in accordance with higher or lower levels (respectively) of IKB and IKKα/β phosphorylation, as expected. The present inventors also found that IKKα/β phosphorylation could be due to Akt activation, as phospho-Akt (P-Akt) levels were increased following paclitaxel treatment, while Gal-3C abrogated this outcome (FIGS. 3A and 3B). Gal-3C alone resulted in reduction of phosphorylated p65, IKKB, IKK, and Akt, compared with controls. To rule out the possibility that alterations in the phosphorylation of p-65, IKB, IKK, or Akt were the results of modulations in the expression levels, total p-65, IKB, IKK, or Akt (phosphorylated and not-phosphorylated forms) were also examined (FIGS. 3A and 3B).

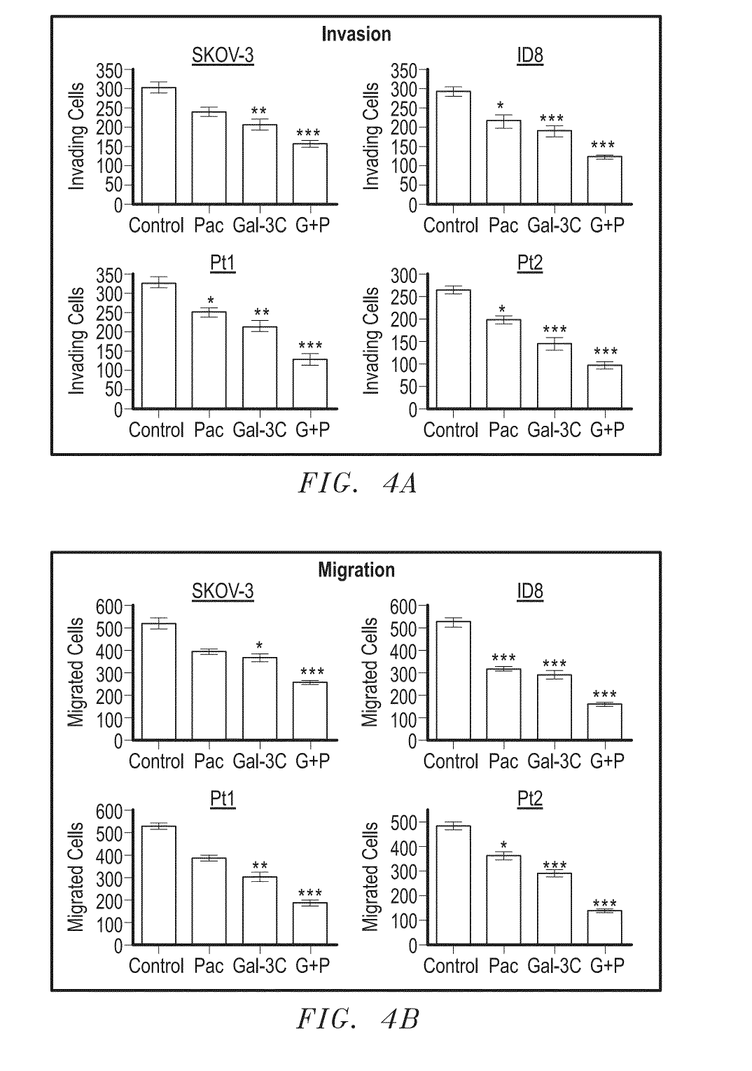

Gal-3C reduced OC invasion and migration alone and in combination with paclitaxel. After 48-hour treatment with 10 μg/mL Gal-3C, 8 nM paclitaxel, or combined drugs, OC cells were tested for in vitro invasivity and motility abilities as described in methods. FIG. 4 shows that Gal-3C alone significantly reduced OC cell invasion and migration compared with controls (one-way ANOVA and Tukey's post-test p<0.05). Paclitaxel resulted in a similar outcome except for SKOV-3 invasion and SKOV-3 and Pt1 migration (FIGS. 4A and 4B). In all of the cell lines tested, Gal-3C significantly hampered invasion and migration (p<0.01 compared with controls), but the combined treatment displayed a more evident inhibition compared with single compounds (FIGS. 4A and 4B).

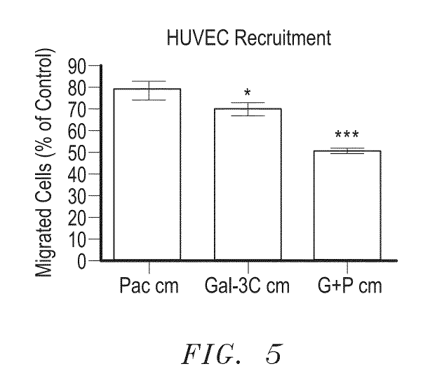

Gal-3C reduced OC ability to recruit vascular endothelial cells. After 48-hour treatments (10 μg/mL Gal-3C, 8 nM paclitaxel, or combined drugs), the conditioned media form SKOV-3 cells was harvested and tested for its ability to induce migration of HUVEC cells in a Transwell assay. FIG. 5 shows that paclitaxel was unable to reduce the production of HUVEC chemotactic factors by OC cells compared with controls (one-way ANOVA and Tukey's post-test p>0.05). Gal-3C showed a significant effect (p<0.05), but the most dramatic inhibition in HUVEC migration was seen by combining Gal-3C and paclitaxel, resulting in 50% (±4%) reduction on average (p<0.001 compared with controls). To test if residual drugs in the conditioned media could inhibit HUVEC migration, a migration assay was performed in the presence of control OC-conditioned medium supplemented with 20% of drug amounts (2 μg/mL Gal-3C, 1.6 nM paclitaxel, and combined drugs). Results (not shown) revealed that residual drugs in the conditioned media did not significantly affect HUVEC chemotaxis.

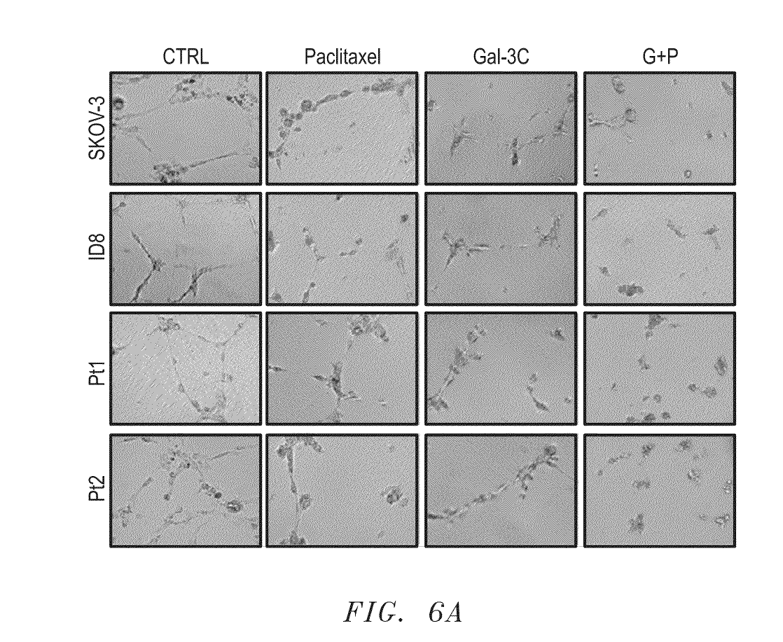

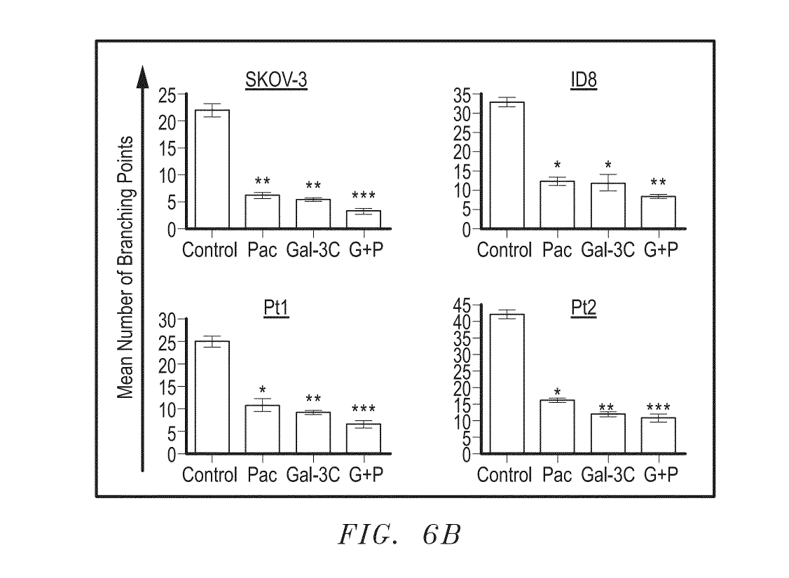

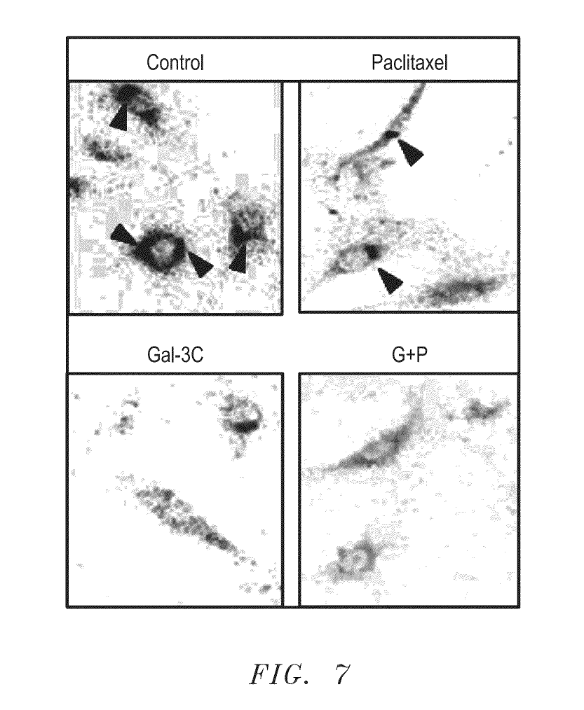

Gal-3C blocked OC cell angiogenic potential and αvβ3 integrin clustering. To study the effects of Gal-3C on OC cells ability to induce the formation of new vessels, an in vitro tubule formation assay was carried out using HUVEC cells stimulated with the conditioned media (20% V/V) of OC cells pre-treated for 48 hours with 10 μg/mL Gal-3C, 8 nM paclitaxel, or a combination of both treatments. Results (FIG. 6A) show that Gal-3C and paclitaxel, alone or in combination, were able to significantly reduce the formation of vessel-like structures by HUVEC cells. The amount of branching points was also calculated through the Wimasis WimTube software, as described hereinabove. FIG. 6B shows that while single as well as combined treatments significantly reduced the average number of branching points in all of the tested OC cells compared with vehicle-treated cells, while no statistically significant difference was found comparing Gal-3C, paclitaxel, or combined regimen with each other (as determined by a Kruskal-Wallis test and a Dunns post-test, level of significance=0.05). A parallel set of experiments were performed using a mixture consisting of the 48-hour conditioned media of each untreated OC cell line (20% V/V) supplemented with 10 μg/mL Gal-3C, 8 nM paclitaxel, or 10 μg/mL Gal-3C+8 nM paclitaxel. Results (not shown) indicate that Gal-3C, paclitaxel, and combined drugs were unable to significantly affect tubule formation, ruling out the possibility that residual drugs in the conditioned media could account for the anti-angiogenic effect observed. This outcome on in vitro angiogenesis was correlated with an evident reduction in the clusters of αvβ3 integrins (FIG. 7). While the conditioned medium of paclitaxel pre-treated OC cells still preserved the ability to induce αvβ3 integrin cluster in HUVEC cells (even if at a lower extent compared with control), Gal-3C (alone or together with paclitaxel) completely abrogated αvβ3 integrin clusters (FIG. 7).

Truncated Galectin-3 was used in the treatment of advanced OC. It is shown herein that Gal-3C negatively and dramatically affects different key features of advanced ovarian tumors, namely fast cell growth, drug resistance, migration, invasion, and angiogenesis.

Flow-cytometry analysis on unpermealized cells revealed that OC expressed Galectin-3. The present inventors have shown the expression of Galectin-3 at the surface of OC cells. Although Galectin-3 is expressed (26) and secreted (27) by OC, and contributes to OC drug resistance (26), it can be variously found either intra- or extra-cellularly (25). Therefore, whether Galectin-3 functions intra-cellularly or extra-cellularly is not known. An immunohistochemical analysis of tumor samples from OC patients indicated that cytoplasmatic Galectin-3 and cyclin-D1 indicate high-risk carcinomas of the ovary (28), but no study has been conducted so far to elucidate the sub-localization of Galectin-3 on the cell surface. Because cell surface molecules, such as integrins, are known targets of Galectins (29), these finding might indicate that OC cells secrete Galectin-3, which binds to membrane proteins. For instance α1β1 integrin, that was shown to be specifically recognized by Galectin-3 (30), is also directly involved in the promotion of OC progression (31).

Once it was shown that Galectin-3 was expressed on the cell surface, the outcome of Gal-3C treatment on cell growth was evaluated. Results indicated that Gal-3C was able to significantly reduce OC cell growth in culture starting from 10 μg/mL and at different extents depending on the cell tested, but irrespectively of Galectin-3 expression levels. Resistance to standard chemotherapeutics such as paclitaxel (32) is evidently a major obstacle to the successful treatment of advanced stage patients (33). The present inventors (25) and others (34-36) have shown that Galectin-3 blockade is a potentially effective strategy to restore sensitivity to chemotherapeutic drugs and apoptotic stimuli in a large variety of human cancers. Therefore, the inventors tested the effects of Gal-3C treatment on paclitaxel response of cultured OC cells, and found that Gal-3C significantly enhanced the growth inhibitory effect of paclitaxel. Mabuchi et al. showed that paclitaxel induced an Aid-initiated activation of NF-κB through IKK and IKBα phosphorylation in OC (22), while the study by Liu et al. indicates that preventing NF-κB activation enhanced OC sensitivity to paclitaxel (37). While not a limitation of the invention, it is possible that Gal-3C interferes with NF-κB stimulation following exposure to paclitaxel. The analysis shown herein demonstrates that paclitaxel triggered the activation of NF-κB through the Akt-IKK-IKB pathway, which was dramatically prevented by the co-administration of Gal-3C. Although the molecular link between Galectin-3 and Akt has still to be elucidated, again without being a limitation of the present invention, it is possible that that Gal-3C blocked the clustering of β1-integrin by Galectin-3 (30), restoring the activity of the Akt inhibitor, PTEN (31). Recently, Yang et al. proposed a model in which NF-κB plays a biphasic role in OC, alternatively acting as a tumor suppressor in primary OC, or as an oncogene in recurrent and drug resistant OC (38). In this scenario, NF-κB blockade alone could be detrimental in primary OC, as Gal-3C was able to reduce the activation of the Akt-IKK-IKB-NF-κB axis even when administered alone.

Nearly 70% of OC patients are diagnosed with metastatic disease, displaying extensive peritoneal metastases, which make available therapies ineffective (39). Therefore, blocking the mechanisms of metastasis is expected to increase the efficacy of surgery and chemotherapy, extending survival. Because of the anatomic position of the ovaries, OC spread requires a direct migration to and invasion into adjacent organs located in the peritoneal space (40). Galectin-3 was reported to induce the migration of monocytes, macrophages, and dendritic cells (41), as well as multiple myeloma cells (25). Accordingly, the present inventors demonstrate herein that Gal-3C blocks the invasion and migration of multiple myeloma (25) and has anti-metastatic activities in a murine model of breast cancer (24). Therefore, the inventors test whether Gal-3C was able to similarly block OC invasion and migration. It was found that while Gal-3C alone markedly hampered OC cell ability to invade migrate, this effect was significantly improved by the co-administration with paclitaxel, which overall showed a blocking effect similar to that of Gal-3C. Little is known of the mechanism involved in paclitaxel-mediated reduction of OC metastatic potential, however the results herein are similar to those showing that the small molecule taxanes blocked OC invasion and migration independently from their cytotoxic activity (42, 43). On the other hand, it is likely that Gal-3C prevented endogenous Galectin-3 from activating adhesion molecules critical for OC cell invasion and motility, such as β1-containing integrins (16, 44) and E-cadherin (45).

Independent studies in OC models and in patients highlight the relevance of the angiogenic process initiated by tumor cells (9, 46). Indeed, tumor vascularization is positively correlated with tumor burden and inversely with progression-free survival and overall survival, often independently of different prognostic factors (46). It has been previously shown that Galectin-3-mediated αvβ3-integrin clustering and activation promote endothelial cell migration and new vessel formation (47). Additionally, the present inventors have reported the Gal-3C cooperated with bortezomib to negatively affect multiple myeloma cell ability to induce angiogenesis in vitro (25). Here a similar outcome was found: exposure to Gal-3C significantly reduced OC cell ability to induce endothelial cell migration and differentiation in vessel-like structures in vitro. As expected (48), this effect was also observed with paclitaxel alone, but it was equally evident following the co-administration of Gal-3C, or Gal-3C alone. Reduced OC angiogenic ability was associated with hampered αvβ3-integrin clustering in endothelial cells, which may contribute to the impairment in endothelial cell migration, invasion and survival (47, 49).

Compared with available standard treatments, an evident advantage of Gal-3C is the very low toxicity profile (24, 25). This work shows that Gal-3C inhibits OC cell proliferation, chemotaxis, and invasiveness, as well as OC-cell induced angiogenesis, and shows for the first time the efficacy of Gal-3C in combination therapy. These data indicate that Gal-3C is a new anti-cancer agent, which may help to answer the need for OC-tailored therapies. The significance of this approach is evident when considering that blocking the complex interactions between tumor cells and the local microenvironment is likely a key future strategy to achieve a better management of this still incurable disease (9). These results demonstrate that the activity of Gal-3C against OC.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Pharoah P D. The potential for risk stratification in the management of ovarian cancer risk. Int J Gynecol Cancer. 2012; 22:S16-7.
2. Shih Ie M, Ho C M, Nakayama K, Salani R. Pathogenesis and new therapeutic targets of ovarian cancer. J Oncol. 2012; 867512:30.
3. Hennessy B T, Coleman R L, Markman M. Ovarian cancer. Lancet. 2009; 374:1371-82.
4. Tsubamoto H, Itani Y, Ito K, Kanazawa R, Toyoda S, Takeuchi S. Phase I I study of interval debulking surgery followed by intraperitoneal chemotherapy for advanced ovarian cancer: A Kansai Clinical Oncology Group study (KCOG9812). Gynecol Oncol. 2012; 10:00803-7.
5. Tagawa T, Morgan R, Yen Y, Mortimer J. Ovarian cancer: opportunity for targeted therapy. J Oncol. 2012; 682480:22.
6. Haslehurst A M, Koti M, Dharsee M, Nuin P, Evans K, Geraci J, et al. EMT transcription factors snail and slug directly contribute to cisplatin resistance in ovarian cancer. BMC Cancer. 2012; 12:1471-2407.
7. Kurman R J, Shih Ie M. The origin and pathogenesis of epithelial ovarian cancer: a proposed unifying theory. Am J Surg Pathol. 2010; 34:433-43.
8. Takahashi Y, Koyanagi T, Suzuki Y, Saga Y, Kanomata N, Moriya T, et al. Vasohibin-2 expressed in human serous ovarian adenocarcinoma accelerates tumor growth by promoting angiogenesis. Mol Cancer Res. 2012; 10:1135-46.
9. Musrap N, Diamandis E P. Revisiting the complexity of the ovarian cancer microenvironment—clinical implications for treatment strategies. Mol Cancer Res. 2012; 10:1254-64.
10. Prat J. New insights into ovarian cancer pathology. Ann Oncol. 2012; 23:x111-x7.
11. Folkman J. Tumor angiogenesis: therapeutic implications. N Engl J Med. 1971; 285:1182-6.
12. Zhang L, Yang N, Park J-W, Katsaros D, Fracchioli S, Cao G, et al. Tumor-derived Vascular Endothelial Growth Factor Up-Regulates Angiopoietin-2 in Host Endothelium and Destabilizes Host Vasculature, Supporting Angiogenesis in Ovarian Cancer. Cancer Research. 2003; 63:3403-12.
13. Zhang L, Yang N, Garcia J R, Mohamed A, Benencia F, Rubin S C, et al.

Generation of a syngeneic mouse model to study the effects of vascular endothelial growth factor in ovarian carcinoma. Am J Pathol. 2002; 161:2295-309.
14. Di Lella S, Sundblad V, Cerliani J P, Guardia C M, Estrin D A, Vasta G R, et al. When galectins recognize glycans: from biochemistry to physiology and back again. Biochemistry. 2011; 50:7842-57.
15. Teichberg V I, Silman I, Beitsch D D, Resheff G. A beta-D-galactoside binding protein from electric organ tissue of Electrophorus electricus. Proc Natl Acad Sci USA. 1975; 72:1383-7.
16. Liu F T, Rabinovich G A. Galectins as modulators of tumour progression. Nat Rev Cancer. 2005; 5:29-41.
17. Kim H J, Jeon H K, Cho Y J, Park Y A, Choi J J, Do I G, et al. High galectin-1 expression correlates with poor prognosis and is involved in epithelial ovarian cancer proliferation and invasion. Eur J Cancer. 2012; 48:1914-21.
18. Turner J G, Dawson J, Sullivan D M. Nuclear export of proteins and drug resistance in cancer. Biochem Pharmacol. 2012; 83:1021-32.
19. Nangia-Makker P, Balan V, Raz A. Galectin-3 binding and metastasis. Methods Mol Biol. 2012; 878:251-66.
20. Kim M K, Sung C O, Do I G, Jeon H K, Song T J, Park H S, et al. Overexpression of Galectin-3 and its clinical significance in ovarian carcinoma. Int J Clin Oncol. 2011; 16:352-8.
21. Guo R X, Qiao Y H, Zhou Y, Li L X, Shi H R, Chen K S. Increased staining for phosphorylated AKT and nuclear factor-kappaB p65 and their relationship with prognosis in epithelial ovarian cancer. Pathol Int. 2008; 58:749-56.
22. Mabuchi S, Ohmichi M, Nishio Y, Hayasaka T, Kimura A, Ohta T, et al. Inhibition of inhibitor of nuclear factor-kappaB phosphorylation increases the efficacy of paclitaxel in in vitro and in vivo ovarian cancer models. Clin Cancer Res. 2004; 10:7645-54.
23. Streetly M J, Maharaj L, Joel S, Schey S A, Gribben J G, Cotter F E. GCS-100, a novel galectin-3 antagonist, modulates MCL-1, NOXA, and cell cycle to induce myeloma cell death. Blood. 2010; 115:3939-48.
24. John C M, Leffler H, Kahl-Knutsson B, Svensson I, Jarvis G A. Truncated galectin-3 inhibits tumor growth and metastasis in orthotopic nude mouse model of human breast cancer. Clin Cancer Res. 2003; 9:2374-83.
25. Mirandola L, Yu Y, Chui K, Jenkins M R, Cobos E, John C M, et al. Galectin-3C Inhibits Tumor Growth and Increases the Anticancer Activity of Bortezomib in a Murine Model of Human Multiple Myeloma. PLoS ONE. 2011; 6: e21811.
26. Oishi T, Itamochi H, Kigawa J, Kanamori Y, Shimada M, Takahashi M, et al. Galectin-3 may contribute to Cisplatin resistance in clear cell carcinoma of the ovary. Int J Gynecol Cancer. 2007; 17:1040-6.
27. Iurisci I, Tinari N, Natoli C, Angelucci D, Cianchetti E, Iacobelli S. Concentrations of galectin-3 in the sera of normal controls and cancer patients. Clin Cancer Res. 2000; 6:1389-93.
28. Brustmann H. Epidermal growth factor receptor expression in serous ovarian carcinoma: an immunohistochemical study with galectin-3 and cyclin D1 and outcome. Int J Gynecol Pathol. 2008; 27:380-9.
29. Ochieng J, Furtak V, Lukyanov P. Extracellular functions of galectin-3. Glycoconj J. 2004; 19:527-
30. Gu M, Wang W, Song W K, Cooper D N, Kaufman S J. Selective modulation of the interaction of alpha 7 beta 1 integrin with fibronectin and laminin by L-14 lectin during skeletal muscle differentiation. J Cell Sci. 1994; 107:175-81.
31. Shen Y, Shen R, Ge L, Zhu Q, Li F. Fibrillar Type I Collagen Matrices Enhance Metastasis/Invasion of Ovarian Epithelial Cancer Via beta1 Integrin and PTEN Signals. Int J Gynecol Cancer. 2012; 22:1316-24.
32. Gillet J P, Calcagno A M, Varma S, Davidson B, Bunkholt Elstrand M, Ganapathi R, et al. Multidrug resistance-linked gene signature predicts overall survival of patients with primary ovarian serous carcinoma. Clin Cancer Res. 2012; 18:3197-206.
33. Cittelly D M, Dimitrova I, Howe E N, Cochrane D R, Jean A, Spoelstra N S, et al. Restoration of miR-200c to ovarian cancer reduces tumor burden and increases sensitivity to paclitaxel. Mol Cancer Ther. 2012; 16:16.
34. Mazurek N, Byrd J C, Sun Y, Hafley M, Ramirez K, Burks J, et al. Cell-surface galectin-3 confers resistance to TRAIL by impeding trafficking of death receptors in metastatic colon adenocarcinoma cells. Cell Death Differ. 2012; 19:523-33.
35. Cheng Y L, Huang W C, Chen C L, Tsai C C, Wang C Y, Chiu W H, et al. Increased galectin-3 facilitates leukemia cell survival from apoptotic stimuli. Biochem Biophys Res Commun. 2011; 412:334-40.
36. Kobayashi T, Shimura T, Yajima T, Kubo N, Araki K, Wada W, et al. Transient silencing of galectin-3 expression promotes both in vitro and in vivo drug-induced apoptosis of human pancreatic carcinoma cells. Clin Exp Metastasis. 2011; 28:367-76.
37. Liu G H, Wang S R, Wang B, Kong B H Inhibition of nuclear factor-kappaB by an antioxidant enhances paclitaxel sensitivity in ovarian carcinoma cell line. Int J Gynecol Cancer. 2006; 16:1777-82.
38. Yang G, Xiao X, Rosen D G, Cheng X, Wu X, Chang B, et al. The biphasic role of N F-kappaB in progression and chemoresistance of ovarian cancer. Clin Cancer Res. 2011; 17:2181-94.
39. Cheung LWT, Leung PCK, Wong AST. Cadherin switching and activation of p120 catenin signaling are mediators of gonadotropin-releasing hormone to promote tumor cell migration and invasion in ovarian cancer. Oncogene. 2010; 29:2427-40.
40. Naora H, Montell D J. Ovarian cancer metastasis: integrating insights from disparate model organisms. Nat Rev Cancer. 2005; 5:355-66.
41. Hsu D K, Chernyaysky A I, Chen H Y, Yu L, Grando S A, Liu F T. Endogenous galectin-3 is localized in membrane lipid rafts and regulates migration of dendritic cells. J Invest Dermatol. 2009; 129:573-83.
42. Belotti D, Rieppi M, Nicoletti M I, Casazza A M, Fojo T, Taraboletti G, et al. Paclitaxel (Taxol(R)) inhibits motility of paclitaxel-resistant human ovarian carcinoma cells. Clin Cancer Res. 1996; 2:1725-30.
43. Westerlund A, Hujanen E, Hoyhtya M, Puistola U, Turpeenniemi-Hujanen T. Ovarian cancer cell invasion is inhibited by paclitaxel. Clin Exp Metastasis. 1997; 15:318-28.
44. Hood J D, Cheresh D A. Role of integrins in cell invasion and migration. Nat Rev Cancer. 2002; 2:91-100.
45. Zhao Q, Barclay M, Hilkens J, Guo X, Barrow H, Rhodes J M, et al. Interaction between circulating galectin-3 and cancer-associated MUC1 enhances tumour cell homotypic aggregation and prevents anoikis. Mol Cancer. 2010; 9:154.
46. Burger R A, Brady M F, Bookman M A, Fleming G F, Monk B J, Huang H, et al. Incorporation of Bevacizumab in the Primary Treatment of Ovarian Cancer. New England Journal of Medicine. 2011; 365:2473-83.
47. Markowska A I, Liu F T, Panjwani N. Galectin-3 is an important mediator of VEGF- and bFGF-mediated angiogenic response. J Exp Med. 2010; 207:1981-93.
48. Belotti D, Vergani V, Drudis T, Borsotti P, Pitelli M R, Viale G, et al. The microtubule-affecting drug paclitaxel has antiangiogenic activity. Clinical Cancer Research. 1996; 2:1843-9.
49. Danhier F, Breton A L, Preat V. RGD-Based Strategies To Target Alpha(v) Beta(3) Integrin in Cancer Therapy and Diagnosis. Molecular Pharmaceutics. 2012; 9:2961-73.
50. Chiriva-Internati M, Grizzi F, Weidanz J A, Ferrari R, Yuefei Y, Velez B, et al. A NOD/SCID tumor model for human ovarian cancer that allows tracking of tumor progression through the biomarker Sp17. J Immunol Methods. 2007; 321:86-93.

What is claimed is:

1. A method for the treatment of an advanced ovarian cancer, comprising:
identifying a patient with advanced ovarian cancer; and
administering to the patient an effective amount of truncated, dominant negative form of Galectin-3 sufficient to reduce the advanced ovarian cancer.

2. The method of claim 1, wherein the truncated, dominant negative form of Galectin-3 is administered by intravenous or intraperitoneal route.

3. The method of claim 1, wherein the ovarian cancer is drug resistant.

4. The method of claim 1, wherein the ovarian cancer is multiple-drug resistant.

5. The method of claim 1, wherein the ovarian cancer is an advanced, refractory ovarian cancer.

6. The method of claim 1, wherein the amount of the truncated, dominant negative form of Galectin-3 is sufficient to reduce at least one of growth, motility, invasion, angiogenesis, or prevents Akt/NF-κB activation in ovarian cancer cells.

7. The method of claim 1, further comprising providing an amount of paclitaxel effective to prevent ovarian cancer cell growth.

8. The method of claim 1, wherein the truncated, dominant negative form of Galectin-3 is provided as a nucleic acid vector having SEQ ID NO.:3, and expressed as SEQ ID NO.: 4.

9. The method of claim 1, wherein the truncated, dominant negative form of Galectin-3 is provided as a polypeptide having SEQ ID NO.: 4.

10. The method of claim 1, wherein the truncated, dominant negative form of Galectin-3 is provided as a nucleic acid in an expression vector that expresses the truncated, dominant negative form of Galectin-3 upon entry into a cell.

11. A method for the treatment of an advanced, refractory ovarian cancer, comprising:
identifying a patient with advanced, refractory ovarian cancer; and
administering to the patient an effective amount of a truncated, dominant negative form of Galectin-3, in combination with paclitaxel, in free or pharmaceutically acceptable salt form to reduce or eliminate the ovarian cancer.

12. The method of claim 11, wherein the truncated, dominant negative form of Galectin-3 is administered by intravenous or intraperitoneal route.

13. The method of claim 11, wherein the ovarian cancer is drug resistant.

14. The method of claim 11, wherein the ovarian cancer is multiple-drug resistant.

15. The method of claim 11, wherein the amount of the truncated, dominant negative form of Galectin-3 is sufficient to reduce at least one of growth, motility, invasion, angiogenesis, or prevents Akt/NF-κB activation in ovarian cancer cells.

16. The method of claim 11, wherein the truncated, dominant negative form of Galectin-3 is provided as a nucleic acid vector having SEQ ID NO.:3, and expressed as SEQ ID NO.: 4.

17. The method of claim 11, wherein the truncated, dominant negative form of Galectin-3 is provided as a polypeptide having SEQ ID NO.:4.

18. The method of claim 11, wherein the truncated, dominant negative form of Galectin-3 is provided as a nucleic acid in an expression vector that expresses the truncated, dominant negative form of Galectin-3 upon entry into a cell.

19. A method of determining the effectiveness of a candidate drug believed to be useful in treating ovarian cancer, the method comprising:
(a) measuring from tissue suspected of having ovarian cancer from a set of patients;
(b) administering a candidate drug to a first subset of the patients, and a placebo to a second subset of the patients, wherein the candidate substance is at least one of a truncated or a dominant negative form of Galectin-3;
(c) repeating step (a) after the administration of the candidate drug or the placebo; and
(d) determining if the candidate drug reduces at least one of the number or proliferation of ovarian cancer cells, reduces at least one of: growth, motility, invasion, or angiogenesis caused by ovarian cancer cells, or prevents Akt/NF-κB activation in ovarian cancer cells that is statistically significant as compared to any reduction occurring in the second subset of patients, wherein a statistically significant reduction indicates that the candidate drug is useful in treating the ovarian cancer.
